(19) United States
(12) Patent Application Publication
(10) Pub. No.: US 2017/0172890 A1
(43) Pub. Date: Jun. 22, 2017

(54) PHOTOCURABLE NAIL COMPOSITIONS CONTAINING DISPERSION OF ACRYLIC POLYMER PARTICLES

(71) Applicant: L'OREAL, Paris (FR)

(72) Inventors: Christopher Michael MACNEILL, Fanwood, NJ (US); XianZhi ZHOU, Millburn, NJ (US); Tsang-Min HUANG, Scotch Plains, NJ (US); Aline Aude GUIMONT, Westfield, NJ (US); Chunhua LI, Hillsborough, NJ (US); Hy Si BUI, Piscataway, NJ (US); Ronni Lynn WEINKAUF, Oradell, NJ (US)

(73) Assignee: L'OREAL, Paris (FR)

(21) Appl. No.: 14/979,263

(22) Filed: Dec. 22, 2015

(51) Int. Cl. A61K 8/81; A61Q 3/02; A61K 8/02

(52) U.S. Cl. CPC A61K 8/8152; A61K 8/0241; A61Q 3/02; A61K 2800/54

(57) ABSTRACT

The invention relates to photocurable compositions comprising at least one dispersion of acrylic polymer particles.

### FIELD OF THE INVENTION

The present invention relates to photocurable nail compositions comprising at least one dispersion of acrylic polymer particles.

### DISCUSSION OF THE BACKGROUND

UV gel compositions typically consist of a layer of base coat for adhesion on the nails, one or more color coats to enhance the color, and a layer of top coat for shine. Each coating needs to be cured with a UV Lamp or UV LED. A UV gel composition set is thus a system that typically contains base coat, color coat and top coat layers. The UV gel composition set's adhesion on the nail and the cohesion among the layers is so strong that it is difficult to remove such composition sets from nails. To remove such UV gel products from nails, it is usually required to soak nails with harsh solvent such as acetone for 20 minutes or more, followed by scraping the product off the nail. Frequent and/or prolonged use of such solvents in this manner and associated scraping can damage nails such as, for example, by making them dry and brittle. At the same time, the removal process is time-consuming.

Further, consumers come in contact with water several times a day during the course of the day, (for example, showers, hand washing, washing dishes, etc.). Such nail compositions sets are susceptible to damage by such frequent contact with water.

U.S. patent application publication no. 2013/0263875 relates to monophasic energy-curable solvent-free compositions which are formulated using at least one energy-curable resin and at least one film-former.

PCT patent application publication no. WO 2015/022438 relates to “novel plasticizers for nail varnish.”

U.S. Pat. Nos. 8,901,199, 6,803,394, 8,263,677 and 8,399,537 also propose photocrosslinkable nail compositions.

It would be desirable to possess a UV gel product which has one or more of the following properties (preferably, all of the following properties): good water-resistance, good wear and/or good adhesion, without prolonging the amount of time needed for removal from nails.

There remains a need for UV gel compositions which are safe and adhere well to nails, and which have some or all of the desired properties discussed above.

### SUMMARY OF THE INVENTION

The present invention relates to photocurable nail compositions comprising of at least one dispersion of acrylic polymer particles.

The present invention also relates to a nail composition set comprising (1) at least one base coat composition; and (2) at least one photocurable color coat composition comprising at least one dispersion of acrylic polymer particles.

The present invention also relates to a nail composition set comprising (1) at least one top coat composition; and (2) at least one photocurable color coat composition comprising at least one dispersion of acrylic polymer particles.

The present invention also relates to a nail composition set comprising (1) at least one base coat composition; (2) at least one photocurable color coat composition comprising at least one dispersion of acrylic polymer particles; and (3) at least one photocurable top coat composition comprising at least one dispersion of acrylic polymer particles.

The present invention further relates to methods for making up and/or protecting nails comprising applying to the nails at least one photocurable nail composition comprising at least one dispersion of acrylic polymer particles.

The present invention further relates to methods for making up and/or protecting nails comprising applying to the nails at least one nail composition set comprising (1) at least one base coat composition; and (2) at least one photocurable color coat composition comprising at least one dispersion of acrylic polymer particles.

The present invention further relates to methods for making up and/or protecting nails comprising applying to the nails at least one nail composition set comprising (1) at least one top coat composition; and (2) at least one photocurable color coat composition comprising at least one dispersion of acrylic polymer particles.

The present invention further relates to methods for making up and/or protecting nails comprising applying to the nails at least one nail composition set comprising (1) at least one base coat composition; (2) at least one photocurable color coat composition comprising at least one dispersion of acrylic polymer particles; and (3) at least one top coat composition comprising at least one dispersion of acrylic polymer particles.

The present invention also relates to methods for improving removal properties of a photocurable nail composition comprising adding at least one dispersion of acrylic polymer particles to the nail composition, resulting in a nail composition having improved removal properties after curing.

The present invention also relates to a kit for a nail composition set comprising at least one photocurable nail composition comprising at least one dispersion of acrylic polymer particles.

The present invention also relates to a kit for a nail composition set comprising (1) at least one base coat composition; and (2) at least one photocurable color coat composition comprising at least one dispersion of acrylic polymer particles.

The present invention also relates to a kit for a nail composition set comprising (1) at least one top coat composition; and (2) at least one photocurable color coat composition comprising at least one dispersion of acrylic polymer particles.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9-11% and “about 2%” means 1.8%-2.2%).

“A” or “an” as used herein means “at least one.”

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Adhesion” as used herein, refers to chemical and/or physical bonding between a coating and a substrate. Good adhesion between nail polish and nail surface should translate to good wear properties on consumers.

“Adhesive agent” or “adhesive” means a polymer that improves chemical and/or physical bonding between a coating and a substrate. In this invention, the adhesive agent improves bonding between compositions and the nail surface or other compositions.

“Removal” or “Easy removal” means the composition may be substantially removed with acetone or other organic solvents not limited to butyl acetate, isopropyl alcohol, ethanol, ethyl acetate, methyl acetate, methyl ethyl ketone, and mixtures thereof, followed by scraping of the composition from the nail.

“Film former”, “film-forming polymer” or “film forming agent” or “co-film former” as used herein means a polymer or resin that leaves a film on the substrate to which it is applied, for example, after a solvent accompanying the film former has evaporated, absorbed into and/or dissipated on the substrate.

“Free” or “devoid” of as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of solvents” means that non-aqueous solvents are preferably omitted (that is 0% by weight), but can be present in the composition at an amount of less than about 0.25% by weight, typically less than about 0.1% by weight, typically less than about 0.05% by weight, based on the total weight of the composition.

“Water free” or “free of water” herein means that water is preferably omitted (that is 0% by weight), but can be present in the composition at an amount of less than about 0.25% by weight, typically less than about 0.1% by weight, typically less than about 0.05% by weight, based on the total weight of the composition.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to nails and evaluating the color of the composition after an extended period of time. For example, the color of a composition may be evaluated immediately following application to nails and these characteristics may then be re-evaluated and compared after a certain amount of time. Further, these characteristics may be evaluated with respect to other compositions, such as commercially available compositions.

“Making up” as used herein means to provide decoration (for example, color) to the nail.

“Protecting” as used herein means to inhibit damage to the nail (for example, chipping) by providing a protective layer on the nail.

“Nails”, “fingernail or “toenail” refers to a human keratinous substrate on a finger or toe which can be treated (decorated) with a single or multiple nail cosmetic compositions.

“Nail treatment system” or “nail composition set” means multiple compositions applied on the surface of nails.

“Nail composition” or “lacquer” or “nail polish” or “nail enamel” or “nail coating” or “nail film” refers to nail enamel usable as a base coat, color coat, top coat, clear coat and protective coat applied on nails separately and/or as a combined application of the above.

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Gloss” in compositions as used herein refers to compositions having with an average gloss, measured at 20°, of greater than or equal to 35, for example 40, preferably 45, 55, 60 or 65, including all ranges and subranges therebetween such as 35-65, 40-65, etc., and/or an average gloss, measured at 60° , of greater than or equal to 65, 70, 75 or 80, including all ranges and subranges therebetween such as 65-80, 65-75, etc.

The term “average gloss” denotes the gloss as it can be measured using a gloss meter, for example by spreading a layer of the composition to be tested, between 50 μm and 150 μm in thickness, on a white Leneta contrast card using an automatic spreader. The deposit is cured under UV-LED lamp for 1 min. The residual tacky layer is wiped off with lint free cotton saturated in alcohol solvent, and then the gloss is measured at 20° using a Byk Gardner gloss meter of reference microTRI-GLOSS. This measurement is repeated at least three times, and the average gloss in GU (gloss units) is the average of the at least three measurements carried out.

The average gloss at 60° is measured in a similar manner, the measurement being carried out at 60° rather than 20°.

“Shine enhancing agent” or “shine increasing agent” in accordance with the present invention means increasing shine or, as the case may be, mitigating or reducing any reduction in shine that may result from the use of corresponding amounts of non-shine enhancing materials.

“Water resistance” as used herein, means resistance of a material (substance) to the penetration of water, which may cause degradation of that material. The method implemented if assessment of this invention is further disclosed

The compositions and methods of the present invention can comprise, consist of, or consist essentially of the essential elements and limitations of the invention described herein, as well as any additional or optional ingredients, components, or limitations described herein or otherwise useful.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

Dispersion of Acrylic Polymer Particles

According to the present invention, nail compositions containing at least one dispersion of acrylic polymer particles are provided. According to the present invention, the dispersions of acrylic polymer particles are dispersions of C1-C4 alkyl (meth)acrylate polymer particles stabilized with stabilizers based on isobornyl (meth)acrylate polymer in a hydrocarbon-based oil. The dispersion of acrylic polymer particles has been previously disclosed in PCT patent application serial no. PCT/EP2014/07800, the entire contents of which is hereby incorporated by reference.

According to preferred embodiments, the polymer of the particles is a C1-C4 alkyl (meth)acrylate polymer. The C1-C4 alkyl (meth)acrylate monomers may be chosen from methyl (meth)acrylate, ethyl (meth)acrylate, n-propyl (meth)acrylate, isopropyl (meth)acrylate, n-butyl (meth)acrylate and tert-butyl (meth)acrylate. Preferably, the monomer is a C1-C4 alkyl acrylate monomer. Preferably, the polymer of the particles is a methyl acrylate and/or ethyl acrylate polymer.

The polymer of the particles may also comprise an ethylenically unsaturated acid monomer or the anhydride thereof, chosen preferably from ethylenically unsaturated acid monomers comprising at least one carboxylic, phosphoric or sulfonic acid function, such as, for example, crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid or acrylamidoglycolic acid, and/or salts thereof. Preferably, the ethylenically unsaturated acid monomer is chosen from (meth)acrylic acid, maleic acid and maleic anhydride. The salts may preferably be chosen from salts of alkali metals, for example sodium or potassium; salts of alkaline-earth metals, for example calcium, magnesium or strontium; metal salts, for example zinc, aluminium, manganese or copper; ammonium salts of formula NH4+; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts.

The polymer of the particles may thus comprise or consist essentially of 80% to 100% by weight of C1-C4 alkyl (meth)acrylate and of 0 to 20% by weight of ethylenically unsaturated acid monomer, relative to the total weight of the polymer.

According to preferred embodiments, the polymer consists essentially of a polymer of one or more C1-C4 alkyl (meth)acrylate monomers.

According to preferred embodiments, the polymer consists essentially of a copolymer of C1-C4 (meth)acrylate and of (meth)acrylic acid or maleic anhydride.

The polymer of the particles may be chosen from, for example: methyl acrylate homopolymers; ethyl acrylate homopolymers; methyl acrylate/ethyl acrylate copolymers; methyl acrylate/ethyl acrylate/acrylic acid copolymers; methyl acrylate/ethyl acrylate/maleic anhydride copolymers; methyl acrylate/acrylic acid copolymers; ethyl acrylate/acrylic acid copolymers; methyl acrylate/maleic anhydride copolymers; and ethyl acrylate/maleic anhydride copolymers.

Preferably, the polymer of the particles is a non-crosslinked polymer.

The polymer of the particles of the dispersion preferably has a number-average molecular weight ranging from about 2000 to about 10,000,000, preferably ranging from about 150,000 to 500,000, including all ranges and subranges therebetween.

The polymer of the particles are preferably present in the dispersion in a content ranging from about 21% to about 58.5% by weight, preferably ranging from about 36% to about 42% by weight, relative to the total weight of the dispersion, including all ranges and subranges therebetween.

The stabilizer is preferably an isobornyl (meth)acrylate polymer chosen from isobornyl (meth)acrylate homopolymer and statistical copolymers of isobornyl (meth)acrylate and of C1-C4 alkyl (meth)acrylate present in an isobornyl (meth)acrylate/C1-C4 alkyl (meth)acrylate weight ratio of greater than 4. Preferably, the weight ratio ranges from about 4.5 to about 19, including all ranges and subranges therebetween.

Preferably, the stabilizer is chosen from, for example: isobornyl acrylate homopolymers; statistical copolymers of isobornyl acrylate/methyl acrylate; statistical copolymers of isobornyl acrylate/methyl acrylate/ethyl acrylate; statistical copolymers of isobornyl methacrylate/methyl acrylate, in the weight ratio described previously.

The stabilizing polymer preferably has a number-average molecular weight ranging from about 10,000 to about 400,000, preferably ranging from about 20,000 to about 200,000, including all ranges and subranges therebetween.

Although not wishing to be bound by any particular theory, it is believed that the stabilizer is in contact with the surface of the polymer particles and thus makes it possible to stabilize these particles at the surface in order to keep these particles in dispersion in the non-aqueous medium of the dispersion.

Preferably, the combination of the stabilizer+polymer of the particles present in the dispersion comprises from about 10% to about 50% by weight of polymerized isobornyl (meth)acrylate, and from about 50% to about 90% by weight of polymerized C1-C4 alkyl (meth)acrylate, relative to the total weight of the combination of the stabilizer+polymer of the particles.

Preferably, the combination of the stabilizer+polymer of the particles present in the dispersion comprises from about 15% to about 30% by weight of polymerized isobornyl (meth)acrylate, and from about 70% to about 85% by weight of polymerized C1-C4 alkyl (meth)acrylate, relative to the total weight of the combination of the stabilizer+polymer of the particles.

The oily medium of the polymer dispersion comprises a hydrocarbon-based oil.

The hydrocarbon-based oil is an oil that is liquid at room temperature (25° C.).

The term “hydrocarbon-based oil” means an oil formed essentially from, or even consisting of, carbon and hydrogen atoms, and optionally oxygen and nitrogen atoms, and not containing any silicon or fluorine atoms. It may contain, for example, alcohol, ester, ether, carboxylic acid, amine and/or amide groups.

The hydrocarbon-based oil may be chosen from, for example:

hydrocarbon-based oils containing from 8 to 14 carbon atoms, preferably:

branched C8-C14 alkanes, for instance C8-C14 isoalkanes of petroleum origin (also known as isoparaffins), for instance isododecane (also known as 2,2,4,4,6-pentamethylheptane), isodecane and, for example, the oils sold under the trade name Isopar or Permethyl,

linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in Examples 1 and 2 of patent application WO 2008/155 059 from the company Cognis, the disclosure of which is hereby incorporated by reference, and mixtures thereof,

short-chain esters (containing from 3 to 8 carbon atoms in total) such as ethyl acetate, methyl acetate, propyl acetate or n-butyl acetate,

hydrocarbon-based oils of plant origin such as triglycerides consisting of fatty acid esters of glycerol, the fatty acids of which may have chain lengths varying from C4 to C24, these chains possibly being linear or branched, and saturated or unsaturated; these oils are especially heptanoic or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion-flower oil and musk rose oil; shea butter; or else caprylic/capric acid triglycerides, for instance those sold by the company Stéarineries Dubois or those sold under the names Miglyol 810®, 812® and 818® by the company Dynamit Nobel,

synthetic ethers containing from 10 to 40 carbon atoms;

linear or branched hydrocarbons of mineral or synthetic origin, such as petroleum jelly, polydecenes, hydrogenated polyisobutene such as Parleam®, squalane and liquid paraffins, and mixtures thereof,

synthetic esters such as oils of formula R1COOR2 in which R1 represents a linear or branched fatty acid residue containing from 1 to 40 carbon atoms and R2 represents an, in particular, branched hydrocarbon-based chain containing from 1 to 40 carbon atoms, on condition that R1+R2≧10, for instance purcellin oil (cetostearyl octanoate), isopropyl myristate, isopropyl palmitate, C12 to C15 alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alkyl or polyalkyl heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,

fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

Preferably, the hydrocarbon-based oil is apolar (formed solely from carbon and hydrogen atoms).

The hydrocarbon-based oil is preferably chosen from hydrocarbon-based oils containing from 8 to 14 carbon atoms, in particular the apolar oils described previously. Preferably, the hydrocarbon-based oil is isododecane.

The polymer particles of the dispersion preferably have an average size, especially a number-average size, ranging from about 50 to about 500 nm, preferably ranging from about 75 to about 400 nm, and preferably ranging from about 100 to about 250 nm, including all ranges and subranges therebetween.

In general, the dispersion according to the invention may be prepared in the following manner, which is given as an example.

The polymerization may be performed in dispersion, i.e. by precipitation of the polymer during formation, with protection of the formed particles with a stabilizer. In a first step, the stabilizing polymer is prepared by mixing the constituent monomer(s) of the stabilizing polymer, with a radical initiator, in a solvent known as the synthesis solvent, and by polymerizing these monomers. In a second step, the constituent monomer(s) of the polymer of the particles are added to the stabilizing polymer formed and polymerization of these added monomers is performed in the presence of the radical initiator.

When the non-aqueous medium is a non-volatile hydrocarbon-based oil, the polymerization may be performed in an apolar organic solvent (synthesis solvent), followed by adding the non-volatile hydrocarbon-based oil (which should be miscible with the said synthesis solvent) and selectively distilling off the synthesis solvent. A synthesis solvent which is such that the monomers of the stabilizing polymer and the free-radical initiator are soluble therein, and the polymer particles obtained are insoluble therein, so that they precipitate therein during their formation, is thus chosen. In particular, the synthesis solvent may be chosen from alkanes such as heptane or cyclohexane.

When the non-aqueous medium is a volatile hydrocarbon-based oil, the polymerization may be performed directly in the oil, which thus also acts as synthesis solvent. The monomers should also be soluble therein, as should the free-radical initiator, and the polymer of the particles obtained should be insoluble therein.

The monomers are preferably present in the synthesis solvent, before polymerization, in a proportion of about 5 to about 20% by weight. The total amount of monomers may be present in the solvent before the start of the reaction, or part of the monomers may be added gradually as the polymerization reaction proceeds. The free-radical initiator is preferably azobisisobutyronitrile or tert-butyl peroxy-2-ethylhexanoate.

The polymerization may be performed at a temperature ranging from about 70 to about 110° C.

The polymer particles are surface-stabilized, when they are formed during the polymerization, by means of the stabilizer.

The stabilization may be performed by any known means, and in particular by direct addition of the stabilizer, during the polymerization. The stabilizer is preferably also present in the mixture before polymerization of the monomers of the polymer of the particles. However, it is also possible to add it continuously, especially when the monomers of the polymer of the particles are also added continuously.

From about 10% to about 30% by weight, preferably from about 15% to about 25% by weight of stabilizer may be used, relative to the total weight of monomers used (stabilizer+polymer of the particles).

The polymer particle dispersion preferably comprises from about 30% to about 65% by weight, preferably from about 40% to about 60% by weight of solids, relative to the total weight of the dispersion.

Preferably, the oily dispersion may comprise a plasticizer, for example, a plasticizer chosen from tri-n-butyl citrate, tripropylene glycol monomethyl ether (INCI name: PPG-3 methyl ether) and trimethyl pentaphenyl trisiloxane (sold under the name Dow Corning PH-1555 HRI Cosmetic Fluid by the company Dow Corning). These plasticizers make it possible to improve the mechanical strength of the polymer film. The plasticizer, if present, may be present in the oily dispersion in an amount ranging from about 5% to about 50% by weight, relative to the total weight of the polymer of the particles.

According to preferred embodiments, the polymer of the particles is a C1-C4 alkyl (meth)acrylate polymer; the stabilizer is an isobornyl (meth)acrylate polymer chosen from isobornyl (meth)acrylate homopolymer and statistical copolymers of isobornyl (meth)acrylate and of C1-C4 alkyl (meth)acrylate present in an isobornyl (meth)acrylate/C1-C4 alkyl (meth)acrylate weight ratio of greater than 4. For these statistical stabilizing copolymers, the defined weight ratio makes it possible to obtain a polymer dispersion that is stable, especially after storage for seven days at room temperature (25° C.).

The dispersions according to the invention consist of particles, which are generally spherical, of at least one surface-stabilized polymer, in a non-aqueous medium.

Preferably, the amount of acrylic polymer particles present in the compositions of the present invention ranges from about 0.1% to about 20% by weight based on total weight of the composition, preferably about 1% to about 10% by weight based on the total weight of the composition, preferably about 5% to about 10% by weight based on the total weight of the composition, including all ranges and subranges therebetween. Preferably, the dispersion of acrylic polymer particles is present in amounts of active material greater than 0.1%, preferably greater than 0.5%, and preferably greater than 1% based on the total weight of the nail composition, including all ranges and subranges in between.

Base Composition for the Nail Composition

According to preferred embodiments, the base composition for the nail compositions of the present invention is a UV gel composition. Examples of such UV gel compositions include, but are not limited to, a photocrosslinkable composition such as disclosed in U.S. patent application publication no. 2015/0306013 (the entire content of which is hereby incorporated by reference) and a fast-curing composition such as disclosed in U.S. patent application publication no. 2015/0139924 (the entire content of which is hereby incorporated by reference). Set forth below are examples of ingredients which can be found in the nail compositions of the present invention, although all of the identified ingredients need not be present.

Photocrosslinkable Compound

According to preferred embodiments, the nail compositions of the present invention comprise at least one photocrosslinkable compound. The term “photocrosslinkable compound” refers to an organic compound suitable for crosslinking under the action of a light ray and/or UV rays, resulting in a crosslinked polymer network.

Urethane (Meth)Acrylate Compound

According to preferred embodiments, the photocrosslinkable compound is at least one photocrosslinkable urethane (meth)acrylate compound. The term “urethane (meth)acrylate compound” refers to any compound comprising at least one urethane function 13 O—C(O)—NH—, also known as a carbamate, and at least one (meth)acrylate function according to the formula

H2C═C(R)—C(O)—O—

where R═H or CH3.

The “urethane” function is also referred to as a “carbamate” function. The urethane (meth)acrylate compound may be chosen from the group consisting of urethane poly(meth)acrylate compounds. According to the present invention, the term “poly(meth)acrylate compound” refers to a (meth)acrylate compound comprising a plurality of (meth)acrylate functions.

In this way, the term “poly(meth)acrylate compound” may refer to a compound comprising at least two methacrylate functions, or at least two acrylate functions, or at least one methacrylate function and at least one acrylate function.

As urethane (meth)acrylate compounds, particular mention may be made of urethane dimethacrylate compounds.

The term “urethane dimethacrylate compound” refers to any compound comprising at least one urethane function —O—C(O)—NH—, and two methacrylate functions according to the formula

H2C═C(CH3)—C(O)—O—.

The term “polyurethane group” refers to a group obtained from polymerizing a mixture of monomers comprising isocyanate functions and monomers.

Particularly preferred urethane (meth)acrylate compounds are those commercially available from Esstech, Inc. (www.esstechinc.com) under the name Exothane such as, for example, Exothane 8, Exothane 9, Exothane 10, Exothane 24, Exothane 26, Exothane 32, Exothane 108, and Exothane 126. The Exothane compounds are elastomers having high conversion values properties leading to improved adhesion. For example, preferred compounds have conversion values of 80% or greater, preferably 85% or greater, preferably 90% or greater, including all ranges and subranges therebetween such as, for example, 83% to 99%, 85% to 99%, 90% to 99%, etc. Further, such compounds preferably have high viscosity, preferably between 8,500 cPs and 1,000,000, preferably between 10,000 cPs and 900,000 cPs, and preferably between 20,000 and 850,000 cPs at 25° C., including all ranges and subranges therebetween. Further, such compounds preferably have low shrinkage stress (MPa), preferably 1.0 or less, preferably 0.5 or less, preferably 0.3 or less, preferably 0.2 or less, including all ranges and subranges therebetween. Further, such compounds preferably have high elongation properties, preferably at least 10%, preferably at least 20%, preferably at least 30%, preferably at least 50%, and as high as 100% or higher, including all ranges and subranges therebetween such as, for example, 10% to 100%, 20% to 100%, etc. Further, such compounds preferably have low tensile strength (N/mm2), preferably less than 30 N/mm2, preferably less than 25 N/mm2, and preferably less than 20 N/mm2.

The at least one photocrosslinkable urethane (meth)acrylate compound is preferably present at a total content greater than or equal to 1% by weight, in relation to the total weight of the composition, advantageously ranging from about 1% to about 80%, preferably from about 5% to about 75%, more preferably from about 10% to about 70%, advantageously from about 25% to about 65% by weight in relation to the total weight of the composition.

(Meth)acrylate Monomer (Ethylenically Unsaturated Monomer)

According to preferred embodiments, the photocrosslinkable compound is at least one photocrosslinkable (meth)acrylate monomer. (Meth)acrylate monomer refers to a compound comprising a single (meth)acrylate function according to the formula H2C═C(R)—C(O)—O—, where R═H or CH3 capable of reacting with other molecules. In various embodiments, the at least one (meth)acrylate monomer may have a molecular weight ranging from 100 to about 300, for example, from about 120 to about 250.

In various embodiments, the at least one (meth)acrylate monomer may be chosen from compounds of general formula (I):

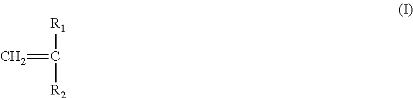

wherein:
R₁ is chosen from hydrogen and C₁-C₃₀ alkyl radicals and R₂ is chosen from —COOM radicals, wherein M is chosen from C₁-C₃₀ straight or branched chain alkyl radicals optionally substituted with at least one hydroxyl group or heterocycle, and from polyalkyleneoxy groups comprising preferably from 2 to 4 units, and from aromatic, alicyclic, and bicyclic rings optionally substituted with at least one substituent chosen from C₁-C₃₀ straight or branched chain alkyl radicals which may be substituted with at least one hydroxyl group. In another embodiment, the at least one (meth)acrylate monomer may be chosen from monomers of formula (I), wherein R₁ is chosen from hydrogen and CH₃, and
R₂ is chosen from —COOM radicals, wherein M is chosen from C₁-C₁₀ straight or branched chain alkyl radicals optionally substituted with at least one hydroxyl group or heterocycle, and from aromatic, alicyclic, and bicyclic rings optionally substituted with at least one substituent chosen from C₁-C₃₀ straight or branched chain alkyl radicals which may be substituted with at least one hydroxyl group.

For example, the (meth)acrylate monomer may be chosen from (meth)acrylate monomers, such as methyl (meth)acrylate (MMA), ethyl (meth)acrylate (EMA), butyl (meth)acrylate (BMA), and polyethylene monomethacrylate such as diethylene glycol monomethacrylate, polypropylene glycol monomethacrylate such as dipropylene glycol monomethacrylate, and isobornyl (meth)acrylate, and tetrahydrofurfuryl (meth)acrylate (THFMA), and hydroxyalkyl (meth)acrylate monomers, such as hydroxypropyl methacrylate (HPMA), hydroxyethyl (meth)acrylate (HEMA), and butoxyethyl (meth)acrylate (BEMA).

Particularly useful for this invention is tetrahydrofurfuryl methacrylate (THFMA) available from Esstech, Inc. (X-958-7466).

Preferably, the (meth)acrylate monomer is present in the composition of the invention in the amount from about 0.01% to about 60% by weight, typically from about 5% to about 45% by weight, more particularly from about 10% to about 35% by weight, including all ranges and subranges there between, all weights being based on the total weight of the composition.

Film Forming Polymer

According to preferred embodiments, the nail compositions of the present invention may optionally further comprise at least one non-photocurable film forming polymer. “Film-forming polymer” refers to a non-photocurable polymer suitable for forming alone (i.e. in the absence of an auxiliary film-forming agent or an external stimulus for example such as UV rays), a film suitable for being isolated, particularly a continuous adherent film, on a substrate, particularly on nails. Preferably, the film forming polymer is selected from the group consisting of radical or polycondensate type synthetic polymers, polymers of natural origin, and mixtures thereof.

Specific examples of suitable film forming polymers include, but are not limited to, polysaccharide derivatives, such as cellulose or guar gum derivatives including nitrocellulose and/or a polysaccharide ester or alkylether such as a polysaccharide consisting of repeat units comprising at least two identical or different rings and having a degree of substitution per saccharide unit between 1.9 and 3, preferably between 2.2 and 2.9, and preferably between 2.4 and 2.8, such as cellulose esters (such as cellulose acetobutyrates or cellulose acetopropionates), cellulose alkylethers (such as ethylcelluloses), and ethylguars.

Specific examples of suitable film forming polymers also include, but are not limited to, alkyd resins, silicone-organic polymer hybrid compounds, polyurethanes, polyvinylbutyrals, and ketone/aldehyde resins, resins from aldehyde condensation products, such as aryl sulfonamide formaldehyde resins such as toluene sulfonamide formaldehyde resin, aryl-sulfonamide epoxy resins or ethyl tosylamide resins.

Preferably, if present, the at least one film forming polymer is present in the nail composition of the present invention in amounts of active material generally ranging from about 2% to about 60%, preferably from about 5% to about 50%, and more preferably from about 8% to about 45%, by weight, based on the total weight of the cosmetic composition, including all ranges and subranges in between.

Photoinitiator

According to preferred embodiments, the nail compositions of the present invention comprise at least one photoinitiator. The photoinitiators suitable for use include those described, for example in “Les photoinitiateurs dans la reticulation des rev tements”, G. Li Bassi, Double Liaison—Chimie des Peintures, No. 361, November 1985, p. 34-41; “Applications industrielles de la polymerisation photoinduite”, Henri Strub, L'Actualite Chimique, February 2000, p. 5-13; and “Photopolymeres: considerations theoriques et reaction de prise”, Marc, J. M. Abadie, Double Liaison—Chimie des Peintures, No. 435-436, 1992, p. 28-34.

Suitable photoinitiators include, but are not limited to, alpha-hydroxyketones, marketed for example under the names DAROCUR® 1173 and 4265, IRGACURE® 184, 2959, and 500 by BASF, and ADDITOL® CPK by CYTEC, alpha.-aminoketones, marketed for example under the names IRGACURE® 907 and 369 by BASF, aromatic ketones marketed for example under the name ESACURE® TZT by LAMBERTI, thioxanthones marketed for example under the name ESACURE® ITX by LAMBERTI, and quinones (these aromatic ketones generally require the presence of a hydrogen donor compound such as tertiary amines and particularly alkanolamines—mention may particularly be made of the tertiary amine ESACURE® EDB marketed by LAMBERTI), alpha-dicarbonyl derivatives of which the most common is benzyl dimethyl ketal marketed under the name IRGACURE® 651 by BASF, and acylphosphine oxides, such as for example bis-acylphosphine oxides (BAPO) marketed for example under the names IRGACURE® 819, 1700, and 1800, DAROCUR® 4265, LUCIRIN® TPO, and LUCIRIN® TPO-L by BASF. Preferably, the photoinitiator is selected from the group consisting of alpha-hydroxyketones, alpha-aminoketones, aromatic ketones preferably associated with a hydrogen donor compound, aromatic alpha-diketones, acylphosphine oxides, and mixtures thereof.

Preferably, the at least one photoinitiator is present in the nail composition of the present invention in amounts of active material generally ranging from about 0.1% to about 10%, preferably from about 1% to about 7%, and more preferably from about 2.5% to about 5%, by weight, based on the total weight of the cosmetic composition, including all ranges and subranges in between.

Solvent

According to preferred embodiments, nail compositions optionally further comprising at least one solvent are provided. Any solvent typically found in nail polish compositions can be used. Suitable solvents include, but are not limited to, organic solvents which are liquid at ambient temperature. Examples of suitable solvents include, but are not limited to, ketones such as methyl ethyl ketone, methyl isobutyl ketone, diisobutyl ketone, isophorone, cyclohexanone or acetone; alcohols, such as ethanol, isopropanol, diacetone alcohol, 2-butoxyethanol or cyclohexanol; glycols, such as ethylene glycol, propylene glycol, pentylene glycol or glycerol; propylene glycol ethers, such as propylene glycol monomethyl ether, propylene glycol monomethyl ether acetate or dipropylene glycol mono(n-butyl) ether; short-chain esters (having a total of 2 to 7 carbon atoms), such as ethyl acetate, methyl acetate, propyl acetate, n-butyl acetate or isopentyl acetate; alkanes, such as decane, heptane, dodecane or cyclohexane; and their mixtures. Most preferred are short-chain esters (having a total of from 2 to 8 carbon atoms).

Preferably, if present, the at least one solvent is present in the nail composition of the present invention in amounts of active material generally ranging from about 0.1% to about 50%, preferably from about 5% to about 40%, and more preferably from about 10% to about 35%, by weight, based on the total weight of the cosmetic composition, including all ranges and subranges in between.

Colorant

According to preferred embodiments, nail compositions further comprising at least one colorant are provided. Any colorant typically found in nail polish compositions can be used. Suitable colorants include, but are not limited to, lipophilic dyes, pigments, pearlescent agents, glitter, and their mixtures.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Preferably, if present, the at least one colorant is present in the nail composition of the present invention in amounts of active material generally ranging from about 0.1% to about 10%, preferably from about 0.25% to about 7%, and more preferably from about 0.5% to about 3.5%, by weight, based on the total weight of the cosmetic composition, including all ranges and subranges in between.

Auxiliaries/Additives

The nail compositions of the present invention may additionally comprise an additive or auxiliary commonly used in cosmetic compositions and known to a person skilled in the art as being capable of being incorporated into a nail polish or varnish composition. Such additives or auxiliaries may be chosen from plasticizers, thickeners, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable. The composition may be in any galenic form normally employed in the cosmetic and dermatological fields which is suitable for topical administration onto nails.

Nail Composition Set

According to the present invention, a nail composition set comprising at least one photocurable color coat and at least one base coat are provided. The nail composition set of the present invention can optionally further comprise at least one primer coat and/or at least one top coat. Any or all of the additional compositions within the nail composition set may also be photocurable, if desired.

For example, a nail composition set comprising at least one primer, at least one base coat, at least one color coat and at least one top coat are provided. However, the primer coat and/or top coat are optional. Thus, nail composition sets comprising at least one primer, at least one base coat and at least one color coat, as well as nail composition sets comprising at least one base coat, at least one color coat and at least one top coat are provided by the present invention.

It should be understood that each coat or layer in the nail composition set, itself, can comprise one or more layers of each composition. Thus, the at least one primer can comprise one or more primer layers; the at least one base coat can comprise one or more base coat layers; the at least one color coat can comprise one or more color coat layers; and the at least one top coat can comprise one or more top coat layers. Preferably, each primer, base coat, color coat and top coat contains three or fewer layers or compositions, more preferably two or fewer layers or compositions, and most preferably a single layer or composition.

According to the present invention, at least one composition of the nail composition set is a photocurable nail composition comprising at least one dispersion of acrylic polymer particles in accordance with the present invention. The other composition(s) of the nail composition set may be any suitable composition for application to nails. For example, the base coat(s) can be an adhesive layer or an undercoat layer; the color coat(s) can be a nail polish composition(s) such as, for example, a standard UV gel composition; the top coat(s) can be an extra shine layer and/or a protective layer, etc.

According to preferred embodiments of the present invention, methods for improving removal properties of a photocurable nail composition comprising adding at least one dispersion of acrylic polymer particles to the nail composition are provided. Such nail compositions possess improved removal properties as compared to the same or similar photocurable nail compositions which do not have at least one dispersion of acrylic polymer particles.

According to such methods, preferably, the amount of dispersion of acrylic polymer particles added to the photocurable nail composition is an amount of active material sufficient to improve removal properties of the nail composition after it has been cured, amounts generally ranging from about 0.1% to about 20% by weight based on total weight of the composition, preferably about 1% to about 10% by weight based on the total weight of the composition, preferably about 5% to about 10% by weight based on the total weight of the composition. Preferably, the dispersion of acrylic polymer particles is present in amounts of active material greater than 0.1%, preferably greater than 0.5%, and preferably greater than 1% based on the total weight of the nail composition, including all ranges and subranges in between.

According to preferred embodiments of the present invention, methods of making up or protecting nails comprising applying to the nails at least one photocurable nail composition comprising at least one dispersion of acrylic polymer particles to nails in an amount sufficient to makeup or protect the nails are provided.

According to preferred embodiments of all method claims discussed above, such methods comprise a) applying at least one coating of a photocurable nail composition of the present invention onto a nail or onto a previously applied composition on a nail (for example, primer), and b) exposing the coated nail to UV or visible light radiation, whereby photocrosslinking of at least one photocrosslinkable compound occurs.

Suitable radiation crosslinking the photocrosslinkable compound has, for example, a wavelength ranging from 210 to 600 nm, preferably from 250 to 420 nm, preferably from 350 to 410 nm. The use of lasers may also effect crosslinking. In one preferred embodiment of the invention methods, a LED lamp or an UV lamp, preferably a mercury vapor lamp, optionally doped with further elements, such as gallium, suitable for modifying the emission spectrum of the light source, can be used. Of course, the exposure time of the deposited coat to radiation is dependent on various factors such as the chemical nature and content of the reactive compounds or the crosslinking density sought. Typically, satisfactory results can be obtained after an exposure time ranging from 10 seconds to 100 minutes, preferably from 30 seconds to 5 minutes.

Before the crosslinking occurs but after application of the nail composition of the present invention, there may be a period for drying the deposited coated layer, the duration of which may vary from 10 seconds to 10 minutes, typically from 30 seconds to 3 minutes. The drying is generally performed in air and at ambient temperature.

According to preferred embodiments of the present invention, a kit for a nail composition set comprising at least one photocurable nail composition comprising at least one dispersion of acrylic polymer particles and at least one additional composition selected from the group consisting of a primer, a base coat, a color coat and a top coat are also provided. Preferably, the kit further comprises one or more of the following: instructions for applying a nail composition of the present invention; instructions for removing a nail composition of the present invention; an abrasive material having a granulometry greater than or equal to 200 μm, preferably less than 300 μm, preferably from 220 μm to 280 μm; and/or a LED lamp or an UV lamp.

The compositions according to the invention can be manufactured by known processes used generally in the cosmetics or dermatological field.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES 1-8

Examples 1-8 are examples of oil dispersions of acrylic polymer particles as found, for example, in PCT Application No. EP2014/07800, the entire contents of which is hereby incorporated by reference.

### EXAMPLE 1

In a first step, 1300 g of isododecane, 337 g of isobornyl acrylate, 28 g of methyl acrylate and 3.64 g of tert-butyl peroxy-2-ethylhexanoate (Trigonox 21S from Akzo) were placed in a reactor. The isobornyl acrylate/methyl acrylate mass ratio is 92/8. The mixture was heated at 90° C. under argon with stirring.

After 2 hours of reaction, 1430 g of isododecane were added to the reactor feedstock and the mixture was heated to 90° C.

In a second step, a mixture of 1376 g of methyl acrylate, 1376 g of isododecane and 13.75 g of Trigonox 21S were run in over 2 hours 30 minutes, and the mixture was left to react for 7 hours. 3.3 litres of isododecane were then added and part of the isododecane was evaporated off to obtain a solids content of 50% by weight.

A dispersion of methyl acrylate particles stabilized with a statistical copolymer stabilizer containing 92% isobornyl acrylate and 8% methyl acrylate in isododecane was obtained.

The oily dispersion contains in total (stabilizer+particles) 80% methyl acrylate and 20% isobornyl acrylate.

The polymer particles of the dispersion have a number-average size of about 160 nm.

The dispersion is stable after storage for 7 days at room temperature (25° C.).

### EXAMPLE 2

A dispersion of polymer in isododecane was prepared according to the preparation method of Example 1, using:

Step 1: 275.5 g of isobornyl acrylate, 11.6 g of methyl acrylate, 11.6 g of ethyl acrylate, 2.99 g of Trigonox 21, 750 g of isododecane; followed by addition, after reaction, of 750 g of isododecane.

Step 2: 539.5 g of methyl acrylate, 539.5 g of ethyl acrylate, 10.8 g of Trigonox 21S, 1079 g of isododecane. After reaction, addition of 2 litres of isododecane and evaporation to obtain a solids content of 35% by weight.

A dispersion in isododecane of methyl acrylate/ethyl acrylate (50/50) copolymer particles stabilized with an isobornyl acrylate/methyl acrylate/ethyl acrylate (92/4/4) statistical copolymer stabilizer was obtained.

The oily dispersion contains in total (stabilizer+particles) 40% methyl acrylate, 40% ethyl acrylate and 20% isobornyl acrylate.

The dispersion is stable after storage for 7 days at room temperature (25° C.).

### EXAMPLE 3

A dispersion of polymer in isododecane was prepared according to the preparation method of Example 1, using:

Step 1: 315.2 g of isobornyl acrylate, 12.5 g of methyl acrylate, 12.5 g of ethyl acrylate, 3.4 g of Trigonox 21, 540 g of isododecane, 360 g of ethyl acetate; followed by addition, after reaction, of 540 g of isododecane and 360 g of ethyl acetate.

Step 2: 303 g of methyl acrylate, 776 g of ethyl acrylate, 157 g of acrylic acid, 11 g of Trigonox 21S, 741.6 g of isododecane and 494.4 g of ethyl acetate. After reaction, addition of 3 litres of an isododecane/ethyl acetate mixture (60/40 weight/weight) and total evaporation of the ethyl acetate and partial evaporation of the isododecane to obtain a solids content of 44% by weight.

A dispersion in isododecane of methyl acrylate/ethyl acrylate/acrylic acid (24.5/62.8/12.7) copolymer particles stabilized with an isobornyl acrylate/methyl acrylate/ethyl acrylate (92/4/4) statistical copolymer stabilizer was obtained.

The oily dispersion contains in total (stabilizer+particles) 10% acrylic acid, 20% methyl acrylate, 50% ethyl acrylate and 20% isobornyl acrylate.

The dispersion is stable after storage for 7 days at room temperature (25° C.).

### EXAMPLE 4

A dispersion of polymer in isododecane was prepared according to the preparation method of Example 1, using:

Step 1: 315.2 g of isobornyl acrylate, 12.5 g of methyl acrylate, 12.5 g of ethyl acrylate, 3.4 g of Trigonox 21, 540 g of isododecane, 360 g of ethyl acetate; followed by addition, after reaction, of 540 g of isododecane and 360 g of ethyl acetate.

Step 2: 145 g of methyl acrylate, 934 g of ethyl acrylate, 157 g of acrylic acid, 12.36 g of Trigonox 21S, 741.6 g of isododecane and 494.4 g of ethyl acetate. After reaction, addition of 3 litres of an isododecane/ethyl acetate mixture (60/40 weight/weight) and total evaporation of the ethyl acetate and partial evaporation of the isododecane to obtain a solids content of 44% by weight.

A dispersion in isododecane of methyl acrylate/ethyl acrylate/acrylic acid (11.7/75.6/12.7) copolymer particles stabilized with an isobornyl acrylate/methyl acrylate/ethyl acrylate (92/4/4) statistical copolymer stabilizer was obtained.

The oily dispersion contains in total (stabilizer+particles) 10% acrylic acid, 10% methyl acrylate, 60% ethyl acrylate and 20% isobornyl acrylate.

The dispersion is stable after storage for 7 days at room temperature (25° C.).

### EXAMPLE 5

A dispersion of polymer in isododecane was prepared according to the preparation method of Example 1, using:

Step 1: 48 g of isobornyl acrylate, 2 g of methyl acrylate, 2 g of ethyl acrylate, 0.52 g of Trigonox 21, 57.6 g of isododecane, 38.4 g of ethyl acetate; followed by addition, after reaction, of 540 g of isododecane and 360 g of ethyl acetate.

Step 2: 98 g of methyl acrylate, 73 g of ethyl acrylate, 25 g of maleic anhydride, 1.96 g of Trigonox 21S, 50.4 g of isododecane and 33.60 g of ethyl acetate. After reaction, addition of 1 litre of an isododecane/ethyl acetate mixture (60/40 weight/weight) and total evaporation of the ethyl acetate and partial evaporation of the isododecane to obtain a solids content of 46.2% by weight.

A dispersion in isododecane of methyl acrylate/ethyl acrylate/maleic anhydride (50/37.2/12.8) copolymer particles stabilized with an isobornyl acrylate/methyl acrylate/ethyl acrylate (92/4/4) statistical copolymer stabilizer was obtained.

The oily dispersion contains in total (stabilizer+particles) 10% maleic anhydride, 30% methyl acrylate, 40% ethyl acrylate and 20% isobornyl acrylate.

The dispersion is stable after storage for 7 days at room temperature (25° C.).

### EXAMPLE 6

A dispersion of polymer in isododecane was prepared according to the preparation method of Example 1, using:

Step 1: 48.5 g of isobornyl methacrylate, 4 g of methyl acrylate, 0.52 g Trigonox 21, 115 g of isododecane; followed by addition, after reaction, of 80 g of isododecane.

Step 2: 190 g of methyl acrylate, 1.9 g of Trigonox 21S, 190 g of isododecane. After reaction, addition of 1 litre of isododecane and partial evaporation of the isododecane to obtain a solids content of 48% by weight.

A dispersion in isododecane of methyl acrylate polymer particles stabilized with an isobornyl methacrylate/methyl acrylate (92/8) statistical copolymer stabilizer was obtained.

The oily dispersion contains in total (stabilizer+particles) 80% methyl acrylate and 20% isobornyl methacrylate.

The dispersion is stable after storage for 7 days at room temperature (25° C.).

### EXAMPLES 7 AND 8

Several oily dispersions of polymethyl acrylate stabilized with a stabilizer containing isobornyl acrylate and optionally methyl acrylate were prepared, according to the procedure of Example 1, by varying the mass ratio of isobornyl acrylate and methyl acrylate and observing the stability of the dispersion obtained as a function of the chemical constitution of the stabilizer.

All the dispersions comprise in total (stabilizer+particles) 80% methyl acrylate and 20% isobornyl acrylate.

### EXAMPLE 7:

Step 1: 50 g of isobornyl acrylate, 0.5 g Trigonox 21, 96 g of isododecane; followed by addition, after reaction, of 80 g of isododecane.

Step 2: 200 g of methyl acrylate, 2 g of Trigonox 21S, 200 g of isododecane. After reaction, addition of 80 g of isododecane and evaporation to obtain a solids content of 31% by weight.

A dispersion in isododecane of polymethyl acrylate particles stabilized with a polyisobornyl acrylate stabilizer was obtained.

### EXAMPLE 8

Step 1: 48.5 g of isobornyl acrylate, 8.5 g of methyl acrylate, 0.57 g Trigonox 21, 115 g of isododecane; followed by addition, after reaction, of 75 g of isododecane.

Step 2: 185.5 g of methyl acrylate, 1.85 g of Trigonox 21S, 185.5 g of isododecane. After reaction, addition of 75 g of isododecane and evaporation to obtain a solids content of 31% by weight.

A dispersion in isododecane of polymethyl acrylate particles stabilized with an isobornyl acrylate/methyl acrylate (85/15) statistical copolymer stabilizer was obtained.

### EXAMPLE 9

### Testing Protocols

Gloss was then determined using a gloss meter. For this determination, a layer of the composition to be tested was spread on a contrast card using an automatic spreader. The layer covered at least the white background of the card and cured for 60 seconds using an LED lamp. Then, the deposit was wiped with isopropanol. Then, gloss was measured at 20° on the white background using a Byk Gardner gloss meter of reference microTRI-GLOSS. This measurement was repeated 3 times, and the average gloss (in gloss units (GU)) is the average of the 3 measurements carried out.

Adhesion properties were also assessed. For this determination, 4 in.×3 in. squares of Vitronails® from IMS Inc. were buffed with OPI 280 grid buffer in one direction. One base coat layer, two color coat layers and one top coat layer were applied and cured for 30 s, 60 s, 60 s and 60 s using a UV-LED lamp. The top coat was wiped with isopropanol to remove the sticky layer. The samples were put in the oven for 6 days at 40° C. to simulated 14 days wear. The adhesion was assessed by determining how difficult it is to remove the product from the Vitronails® substrate using a spatula. The adhesion/removal properties were assessed from 1 to 5, where 5 meant very easy to remove and 1 very difficult.

The four UV Gel top coat compositions were assessed for their removal properties after curing. The top coat composition drawn down using a 3MIL draw down bar and immediately cured for 60 s using a UV-LED lamp. The top coat was wiped with isopropanol after curing to remove the sticky layer. A 1 cm×1 cm cotton pad saturated with 0.3 mL of acetone was allowed to sit on the surface of the top coat composition while covered for 5 minutes. After 5 minutes, the top coat compositions were poked using a cuticle pusher to determine how easily they could be pierced. A ranking of 1 to 5 was assessed, with 5 meaning very easy to pierce and 1 meaning very difficult to pierce.

### EXAMPLE 10

Different weight percentages of the dispersion of acrylic polymer particles were added to a top coat composition (Inventive 1, 2, 3) and compared to the top coat composition without any dispersion of acrylic polymer particles (Comparative 1). The shine, removal and adhesion properties of each inventive composition were tested. The inventive formulations that contained the dispersion of acrylic polymer particles showed the best removal properties. In addition, the inventive compositions displayed excellent shine at 2%, 5% & 10%, particularly at 10% by weight, without sacrificing the adhesive properties of the composition.

| | Compar- | Inven- | Inven- | Inven- |
|---|---|---|---|---|
| INCI name | ative 1 | tive 1 | tive 2 | tive 3 |
| BIS-HEMA IPDI | 60.22 | 59.02 | 57.2 | 54.2 |
| DI-HEMA | 13.22 | 12.96 | 12.56 | 11.9 |
| TRIMETHYLHEXYL | | | | |
| DICARBAMATE | | | | |
| TETRAHYDROFURFURYL | 20.56 | 20.14 | 19.54 | 18.5 |
| METHACRYLATE | | | | |
| BUTYL ACETATE | 2 | 1.96 | 1.9 | 1.8 |
| (and) ACRYLATES | | | | |
| COPOLYMER | | | | |
| OIL DISPERSION | 0 | 2 | 5 | 10 |
| ETHYL | 4 | 3.92 | 3.8 | 3.6 |
| TRIMETHYLBENZOYL | | | | |
| PHENYLPHOSPHINATE | | | | |
| Total | 100.0 | 100.0 | 100.0 | 100.0 |
| Gloss @ 20° (GU) | 67.6 | 50.3 | 65.7 | 72.2 |
| Acetone removal | 1 | 1 | 3 | 4 |
| Adhesion | 5 | 5 | 4.5 | 4.5 |

### EXAMPLE 11

Different weight percentages of the dispersion of acrylic polymer particles were added to a color coat composition (Inventive 4, 5, 6) and compared to the color coat composition without any dispersion of acrylic polymer particles (Comparative 2). The shine, removal and adhesion properties of each inventive composition were tested. Both the shine and removal properties improved without sacrificing the adhesion properties of the composition.

| | Compar- | Inven- | Inven- | Inven- |
|---|---|---|---|---|
| INCI name | ative 2 | tive 4 | tive 5 | tive 6 |
| BIS-HEMA IPDI | 51.22 | 50.2 | 48.66 | 46.1 |
| DI-HEMA | 14.06 | 13.78 | 13.36 | 12.65 |
| TRIMETHYLHEXYL | | | | |
| DICARBAMATE | | | | |
| TETRAHYDROFURFURYL | 21.3 | 20.88 | 20.23 | 19.17 |
| METHACRYLATE | | | | |
| DI-HEMA | 5 | 4.9 | 4.75 | 4.5 |
| TRIMETHYLHEXYL | | | | |
| DICARBAMATE | | | | |
| BUTYL ACETATE | 2 | 1.96 | 1.9 | 1.8 |
| (and) ACRYLATES | | | | |
| COPOLYMER | | | | |
| RED 6 LAKE | 0.58 | 0.57 | 0.55 | 0.52 |
| RED 7 LAKE | 0.7 | 0.68 | 0.66 | 0.63 |
| TITANIUM DIOXIDE | 0.24 | 0.23 | 0.23 | 0.22 |
| (and) OXIDIZED | | | | |
| POLYETHYLENE | | | | |
| CELLULOSE | 0.4 | 0.39 | 0.38 | 0.36 |
| ACETATE BUTYRATE | | | | |
| STEARALKONIUM | 0.5 | 0.49 | 0.48 | 0.45 |
| HECTORITE | | | | |
| OIL DISPERSION | 0 | 2 | 5 | 10 |
| ETHYL | 4 | 3.92 | 3.8 | 3.6 |
| TRIMETHYLBENZOYL | | | | |
| PHENYLPHOSPHINATE | | | | |
| Total | 100.0 | 100.0 | 100.0 | 100.0 |
| Acetone removal | 3 | 3 | 5 | 5 |
| Adhesion | 4 | 4 | 4 | 3.5 |

The addition of dispersion of acrylic polymer particles helped the top coat and color coat formulas obtain high shine and improved removal compared to the Comparative formulas 1 and 2. We found that dispersion of acrylic polymer particles caused the viscosity of our formulations to increase dramatically.

### EXAMPLE 12

### Solvent Optimization

Solvent was added to the formulations below containing the dispersion of acrylic polymer particles (Inventive 7, 8, 9) to determine an optimum synergy.

| | Compar- | Inven- | Inven- | Inven- |
|---|---|---|---|---|
| INCI name | ative 1 | tive 7 | tive 8 | tive 9 |
| BIS-HEMA IPDI | 60.22 | 54.2 | 53 | 51.19 |
| DI-HEMA | 13.22 | 11.9 | 11.63 | 11.24 |
| TRIMETHYLHEXYL | | | | |
| DICARBAMATE | | | | |
| TETRAHYDROFURFURYL | 20.56 | 18.5 | 18.09 | 17.48 |
| METHACRYLATE | | | | |
| BUTYL ACETATE | 2 | 1.8 | 1.76 | 1.7 |
| (and) ACRYLATES | | | | |
| COPOLYMER | | | | |
| OIL DISPERSION | 0 | 10 | 12 | 15 |
| ETHYL | 4 | 3.6 | 3.52 | 3.4 |
| TRIMETHYLBENZOYL | | | | |
| PHENYLPHOSPHINATE | | | | |
| Total | 100.0 | 100.0 | 100.0 | 100.0 |
| Acetone removal | 1 | 4 | 4 | 4 |
| Adhesion | 5 | 45 | 45 | 4 |

By adding solvent to the formulas, the viscosity decreased and removal and shine continued to increase to give us an optimum formulation.

| INCI name | Solvent (wt %) | Shine (Gus, 20o) | Removal |
|---|---|---|---|
| Comparative 1 | X | 67.6 ± 0.5 | 1 |
| Inventive 7 | 7 | 75.5 ± 0.2 | 4 |
| | 10 | 79.7 ± 0.7 | 5 |
| Inventive 8 | 7 | 80.1 ± 0.1 | 4 |
| | 10 | 79.6 ± 0.8 | 5 |
| | 12 | 78.4 ± 0.5 | 5 |
| Inventive 9 | 10 | 74.9 ± 1.5 | 5 |
| | 12 | 77.4 ± 1.3 | 5 |

1. A nail composition comprising at least one photocrosslinkable compound, at least one photoinitiator and at least one dispersion of acrylic polymer particles, wherein the composition is photocurable, and wherein the at least one dispersion of acrylic polymer particles comprises particles of a C1-C4 alkyl (meth)acrylate polymer and at least one stabilizer selected from the group consisting of isobornyl (meth)acrylate homopolymers and statistical copolymers of isobornyl (meth)acrylate and of C1-C4 alkyl (meth)acrylate present in an isobornyl (meth)acrylate/C1-C4 alkyl (meth)acrylate weight ratio of greater than 4.

2. The nail composition of claim 1, wherein the at least one dispersion of acrylic polymer particles is present in an amount ranging from 0.1% to 20% by weight with respect to the total weight of the composition.

3. (canceled)

4. The nail composition of claim 1, wherein the polymer of the particles is a methyl acrylate and/or ethyl acrylate polymer.

5. The nail composition of claim 1, wherein the polymer of the particles comprises an ethylenically unsaturated acid monomer or the anhydride thereof.

6. The nail composition of claim 5, wherein the polymer of the particles comprises from 80% to 100% by weight of C1-C4 alkyl (meth)acrylate and from 0 to 20% by weight of ethylenically unsaturated acid monomer, relative to the total weight of the polymer.

7. The nail composition of claim 1, wherein the polymer of the particles is at least one selected from the group consisting of: methyl acrylate homopolymers; ethyl acrylate homopolymers; methyl acrylate/ethyl acrylate copolymers; methyl acrylate/ethyl acrylate/acrylic acid copolymers; methyl acrylate/ethyl acrylate/maleic anhydride copolymers; methyl acrylate/acrylic acid copolymers; ethyl acrylate/acrylic acid copolymers; methyl acrylate/maleic anhydride copolymers; and ethyl acrylate/maleic anhydride copolymers.

8. The nail composition of claim 1, wherein the polymer particles have an average size ranging from 100 nm to 250 nm.

9. The nail composition of claim 1, wherein the stabilizer is at least one selected from the group consisting of: isobornyl acrylate homopolymers; statistical copolymers of isobornyl acrylate/methyl acrylate; statistical copolymers of isobornyl acrylate/methyl acrylate/ethyl acrylate; and statistical copolymers of isobornyl methacrylate/methyl acrylate.

10. A nail composition set comprising the nail composition of claim 1.

11. (canceled)

12. (canceled)

13. (canceled)

14. The nail composition of claim 1, wherein the at least one dispersion of acrylic polymer is a dispersion of C1-C4 alkyl (meth)acrylate polymer particles stabilized with a stabilizer based on isobornyl (meth)acrylate polymer in a hydrocarbon oil.

15. The nail composition of claim 14, wherein the at least one dispersion of acrylic polymer particles is present in an amount ranging from 0.1% to 20% by weight with respect to the total weight of the composition
